(19) United States
(12) Patent Application Publication
(10) Pub. No.: US 2007/0269539 A1
(43) Pub. Date: Nov. 22, 2007

(54) Ginkgo Biloba Extract as a Treatment for Therapeutic-Induced Neurotoxicity

(75) Inventors: John L. Marshall, Arlington, VA (US); Vassilios Papadopoulos, North Potomac, MD (US)

Correspondence Address: CLARK & ELBING LLP, 101 FEDERAL STREET, BOSTON, MA, 02110, US

(73) Assignee: GEORGETOWN UNIVERSITY, WASHINGTON, DC (US)

(21) Appl. No.: 10/555,322

(22) PCT Filed: May 3, 2004
(86) PCT No.: PCT/US04/13609

§ 371 Date: Mar. 30, 2007

Related U.S. Application Data
(63) Continuation-in-part of application No. 60/467,647, filed on May 2, 2003.

(51) Int. Cl. A61K 36/16; A61K 38/16; A61K 38/13; A61K 31/7034

(52) U.S. Cl. 424/752; 514/11; 514/35; 514/192; 514/200; 514/8; 514/253.08

(57) ABSTRACT

The invention features methods for treating neurotoxicity associated with therapeutic agents, e.g., chemotherapeutic agents, and compositions and kits for use therein. The methods employ an extract of Ginkgo biloba to mitigate the neurotoxic effects of the therapeutic agents.

### BACKGROUND OF THE INVENTION

The invention relates to the fields of treatments for neurotoxicity and pharmaceutical compositions.

Many therapeutic agents for the treatment of conditions or diseases, e.g., cancer, cause unwanted neurological side effects that may limit the use of the agent. Often there is no treatment for these side effects, and after a threshold of treatment is reached, patients undergoing therapy may have to discontinue use of a particular drug for a period of time or all together. Such interruptions may adversely affect the treatment of the patient, for example, by requiring the use of less effective agents albeit with fewer side effects.

In one example, oxaliplatin is an important agent for the treatment of advanced colon cancer, a disease affecting 50,000 patients in the United States annually. The use of oxaliplatin will be extended to a broader group of patients including other gastrointestinal cancers and ovarian cancer. Overall, it is a well-tolerated drug with the exception of significant neurotoxicity. This neurotoxicity is the one major side effect which limits the use of the compound, and it is a common reason for discontinuation of the drug in a patient, even when the drug is still controlling the cancer. Currently, patients are able to receive only 5-6 months of therapy before the neurotoxicity becomes severe and forces the discontinuation of the treatment.

One attempt at reversing the neurotoxicity of oxaliplatin involves a complicated, time consuming intravenous infusion of electrolytes. While the data suggests this process helps, it appears to benefit only 10-20% of patients and adds significantly to the cost and time of treatment for patients.

Accordingly, methods for treating neurotoxicity associated with therapeutic agents that may have a positive impact on both the quality of life and possibly survival of patients, e.g., with advanced cancer, are needed.

### SUMMARY OF THE INVENTION

The invention features methods for treating neurotoxicity associated with therapeutic agents, e.g., chemotherapeutic agents, and compositions and kits for use therein. The methods employ an extract of Ginkgo biloba to mitigate the neurotoxic effects of the therapeutic agents.

In one aspect, the invention features a method of treating therapeutic-induced neurotoxicity in a patient including administering to the patient a therapeutically effective amount of an extract of Ginkgo biloba (e.g., EGb 761, IPS200, LI1379, LI1370, BN 52063, PN246, Geriaforce®, or ZGE). Additional Ginkgo extracts are described herein. In one embodiment, the patient is diagnosed with therapeutic-induced neurotoxicity prior to administering the Ginkgo extract. The method desirably reduces the therapeutic-induced neurotoxicity. The neurotoxicity may be induced by a therapeutic agent as described herein. The method may further include administering antioxidants (e.g., amifostine, alpha-lipoic acid, sodium thiosulfate, diethyldithiocarbamate, 4-methylthiobenzoic acid, L- and D-methionine, salicylate, or glutathione), neurotrophic factors (e.g., nerve growth factor, neurotrophin-3, neurotrophin-4/5, brain-derived neurotrophic factor, and glial-derived neurotrophic factor), melanocortins (e.g., adrenocorticotropin (ACTH), alpha, beta and gamma-melanocyte-stimulating hormones, or Org2766), glutamate, calcium-magnesium infusions, antiepileptic drugs (e.g., carbamazepine or gabapentin), insulin-like growth factor I, or a combination of two, three, four or more thereof. When one or more additional compounds are included to treat neurotoxicity, the combination of the one or more compounds and the extract of Ginkgo biloba is desirably administered in a therapeutically effective amount.

In one embodiment, a patient treated by the above method is suffering from breast cancer, colon cancer, Hodgkin's disease, Kaposi's sarcoma, Letterer-Siwe disease, leukemia, lung cancer, lymphoma, melanoma, ovarian, non-small-cell lung cancer, pancreatic cancer, stomach cancer, or uterine cancer.

The Ginkgo extract is administered, for example, before and/or during and/or after administration of a therapeutic agent to said patient. Administration of the Ginkgo extract may also be alternated with administration of the therapeutic agent.

The method desirably treats neurotoxicity including pain, lack of mobility, ataxia, numbness, tingling, weakness in limbs, nystagmus, dizziness, dysmetria, dysarthria, dysdiadocholinesia, somnolence, seizure, altered personality, areflexia, constipation, hoarseness, orthostatic hypotension, gait disorders, stupor, coma, lethargy, confusion, depression, hallucinations, myoclonus, decreased vibratory sensation, decreased deep tendon reflex, hypersensitivity to temperature (e.g., hot or cold), paresthesias, or a combination thereof.

The invention further features a pharmaceutical composition including an extract of Ginkgo biloba (e.g., EGb 761, IPS200, LI1379, LI1370, BN 52063, PN246, Geriaforce®, or ZGE) and a therapeutic agent, e.g., oxaliplatin. Other exemplary extracts and therapeutic agents are described herein. The composition is useful, for example, in a treatment of cancer. The composition may also include antioxidants (e.g., amifostine, alpha-lipoic acid, sodium thiosulfate, diethyldithiocarbamate, 4-methylthiobenzoic acid, L- and D-methionine, salicylate, or glutathione), neurotrophic factors (e.g., nerve growth factor, neurotrophin-3, neurotrophin-4/5, brain-derived neurotrophic factor, and glial-derived neurotrophic factor), melanocortins (e.g., adrenocorticotropin (ACTH), alpha, beta and gamma-melanocyte-stimulating hormones, or Org2766), glutamate, calcium-magnesium infusions, antiepileptic drugs (e.g., carbamazepine or gabapentin), insulin-like growth factor I, or a combination thereof. In addition, the composition may contain a pharmaceutically acceptable carrier. In various embodiments, the therapeutic agent is present in a therapeutically effective amount for the treatment of an underlying condition (e.g., cancer, infectious disease, arrhythmia, hyperlipidemia, or hyperactive immune response). The extract of Ginkgo biloba, either alone or in combination with additional compounds for the treatment of neurotoxicity as described herein, is also desirably present in a therapeutically effective amount to treat neurotoxicity caused by the therapeutic agent.

In another aspect, the invention features a kit including a therapeutic agent, e.g., oxaliplatin, and an extract of Ginkgo biloba, e.g., EGb 761, IPS200, LI1379, LI1370, BN 52063, PN246, Geriaforce®, or ZGE. The kit may further include labeling for use of the kit in a treatment for therapeutic-induced neurotoxicity. Exemplary Ginkgo extracts and therapeutic agents are described herein. The kit may also include antioxidants (e.g., amifostine, alpha-lipoic acid, sodium thiosulfate, diethyldithiocarbamate, 4-methylthiobenzoic acid, L- and D-methionine, salicylate, or glutathione), neurotrophic factors (e.g., nerve growth factor, neurotrophin-3, neurotrophin-4/5, brain-derived neurotrophic factor, and glial-derived neurotrophic factor), melanocortins (e.g., adrenocorticotropin (ACTH), alpha, beta and gamma-melanocyte-stimulating hormones, or Org2766), glutamate, calcium-magnesium infusions, antiepileptic drugs (e.g., carbamazepine or gabapentin), insulin-like growth factor I, or a combination thereof. In various embodiments, the therapeutic agent is present in a therapeutically effective amount for the treatment of an underlying condition (e.g., cancer, infectious disease, arrhythmia, hyperlipidemia, or hyperactive immune response). The extract of Ginkgo biloba, either alone or in combination with additional compounds for the treatment of neurotoxicity, as described herein, is also desirably present in a therapeutically effective amount to treat neurotoxicity caused by the therapeutic agent.

In various embodiments of any of the above aspects, the therapeutic agent is an immunosuppressant, an antibiotic, an antiarrhythmic agent, an antilipidemic agent, a chemotherapeutic agent, or a combination thereof. Classes of antibiotic agents contemplated by the invention include, without limitation, sulfonamides, tetracyclines, aminoglycosides, tetracyclines, polymyxins, beta lactams, carbapenems, cephalosporins, monobactams, fluoroquinolones, and combinations thereof. Exemplary therapeutic agents include cyclosporine, tacrolimus, chloramphenicol, chloroquine, isoniazid, metronidazole, nitrofurantoin, caprolactam, rifampin, ethionamide, cycloserine, erythromycin, colistin, vancomycin, ethambutol, lincomycin, clindamycin, penicillin, imipenem, cefepime, ceftazidime, cefazolin, cefmetazole, benzylpenicillin, trovafloxacin, ciprofloxacin, levofloxacin, ofloxacin, amiodarone, pyridoxine, bezafibrate, clofibrate, almitrine bimesylate, thalidomide, colchicine, disulfiram, phenyloin, dapsone, sodium aurothiomalate, and combinations thereof. Chemotherapeutic agents may be, for example, alkylating agents, antimetabolite agents, antimicrotubule agents, antimiotic agents, antitumor antibiotics, or combinations thereof. Examples of chemotherapeutic agents include adriamycin, L-asparaginase, BBR3464, carboplatin, chlorambucil, cisplatin, cytarabine, doxetacel, doxorubicin, etoposide, 5-fluorouracil, gemcitabine, hexamethamelemine, ifosamide, IL-2, interferon, JM216, lorazepam, misonidazole, mitotane, nedaplatin, oxaliplatin, pamidronate, pentostatin, plicamycin, procarbazine, SPI-77, suramin, a taxane (e.g., paclitaxel), topotecan, vinblastine, vincristine, vindesine, vinorelbine, xeloda, ZD0473, and combinations thereof.

Desirable therapeutic agents of the invention include cyclosporine, tacrolimus, chloramphenicol, chloroquine, isoniazid, metronidazole, nitrofurantoin, caprolactam, rifampin, ethionamide, cycloserine, erythromycin, colistin, vancomycin, ethambutol, lincomycin, clindamycin, penicillin, imipenem, cefepime, ceftazidime, cefazolin, cefmetazole, benzylpenicillin, trovafloxacin, ciprofloxacin, levofloxacin, ofloxacin, amiodarone, pyridoxine, bezafibrate, clofibrate, almitrine bimesylate, thalidomide, colchicine, disulfiram, phenyloin, dapsone, sodium aurothiomalate, L-asparaginase, carboplatin, chlorambucil, cytarabine, etoposide, hexamethamelemine, ifosamide, IL-2, interferon, lorazepam, misonidazole, mitotane, oxaliplatin, pamidronate, pentostatin, plicamycin, procarbazine, suramin, topotecan, vinblastine, vincristine, vindesine, vinorelbine, xeloda, JM216, ZD0473, BBR3464, SPI-77, nedaplatin, and combinations thereof.

By “chemotherapeutic agent” is meant a drug used alone or in combination to treat cancer. Exemplary classes of chemotherapeutic agents include alkylating agents, antimetabolites, antimicrotubules, antimiotics, and antitumor antibiotics. Examples of chemotherapeutic agents include, without limitation, adriamycin, L-asparaginase, carboplatin, chlorambucil, cisplatin, cytarabine, doxetacel, doxorubicin, etoposide, 5-fluorouracil, gemcitabine, hexamethamelemine, ifosamide, IL-2, interferon, lorazepam, misonidazole, mitotane, oxaliplatin, paclitaxel, pamidronate, pentostatin, plicamycin, procarbazine, suramin, topotecan, vinblastine, vincristine, vindesine, vinorelbine, and xeloda.

By “extract of Ginkgo biloba” or “Ginkgo extract” is meant a composition containing at least one of the individual compounds which can be obtained by extraction from the Ginkgo biloba tree, and in particular a flavonoid compound or a terpene such as a ginkgolide or a bilobalide, or a mixture thereof. Desirable Ginkgo extracts for use in the present invention are useful for treating neurotoxicity. EGb 761, IPS200, LI1379, LI1370, BN 52063, PN246, Geriaforce®, and ZGE are exemplary Ginkgo extracts. Other Ginkgo extracts are known in the art as described, for example, in U.S. Pat. Nos. 4,981,688, 5,322,688, 5,389,370, 5,399,348, 5,512,286, 5,637,302, 5,972,952, 6,030,621, 6,086,883, 6,221,356, 6,274,621, 6,328,999, 6,447,819, and 6,475,534, and International Publication Nos. WO97/17068, WO99/64028, WO01/12208, and WO01/75181, each of which is hereby incorporated by reference.

By “neurotoxicity” is meant damage to the central nervous system or peripheral nervous system. Examples of neurotoxic effects include, without limitation, pain, lack of mobility, ataxia, numbness, tingling, weakness in limbs, nystagmus, dizziness, dysmetria, dysarthria, dysdiadocholinesia, somnolence, seizure, altered personality, areflexia, constipation, hoarseness, orthostatic hypotension, gait disorders, stupor, coma, lethargy, confusion, depression, hallucinations, myoclonus, decreased vibratory sensation, decreased deep tendon reflex, hypersensitivity to temperature (e.g., hot or cold), and paresthesias.

By a “reduction” in neurotoxicity is meant an alleviation or elimination of the systems of neurotoxicity. A reduction can be measured, for example, by physical examination, by examination of a patient's medical history, or by a test of neurological function.

By a “therapeutic-induced” condition is meant a condition, e.g., neurotoxicity, that occurs as a result of a treatment with a therapeutic agent, e.g., a chemotherapeutic agent.

By “therapeutically effective amount” is meant an amount of a pharmaceutical composition, containing one or more active compounds, sufficient to produce a preventative, healing, curative, stabilizing, or ameliorative effect in the treatment of a disease or condition, e.g., neurotoxicity.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder.

Other features and advantages of the invention will be apparent from the following description and the claims.

### DETAILED DESCRIPTION OF THE INVENTION

The invention features methods and compositions for treating neurotoxicity associated with drug therapy.

Therapeutic-Induced Neurotoxicity

Neurotoxicity is an unfortunate and often dose-limiting side effect of therapeutic agents. Common therapeutic agents that cause neurotoxic side effects include chemotherapeutic agents and other agents such as immunosuppressants (e.g., cyclosporine and tacrolimus), antibiotics (e.g., chloramphenicol, chloroquine, isoniazid, metronidazole, nitrofurantoin, caprolactam, rifampin, ethionamide, cycloserine, erythromycin, sulfonamides, tetracyclines, colistin, aminoglycosides, vancomycin, ethambutol, tetracyclines, polymyxins, lincomycin, clindamycin, beta lactams (e.g., carbapenems, cephalosporins, monobactams, penicillin, imipenem, cefepime, ceftazidime, cefazolin, cefmetazole, and benzylpenicillin), and fluoroquinolones (e.g., trovafloxacin, ciprofloxacin, levofloxacin, and ofloxacin)), antiarrhythmic agents (e.g., amiodarone), antilipidemic agents (e.g., bezafibrate and clofibrate), and other drugs (e.g., almitrine bimesylate, thalidomide, colchicine, disulfiram, phenyloin, pyridoxine, dapsone, and sodium aurothiomalate). Examples of chemotherapeutic agents include, without limitation, platinum-containing compounds (such as carboplatin, cisplatin, oxaliplatin, JM216, ZD0473, BBR3464, SPI-77, and nedaplatin), taxanes (such as paclitaxel), vinca alkaloids (such as vincristine, vinblastine, vindesine, and vinorelbine), adriamycin, L-asparaginase, chlorambucil, cytarabine, doxetacel, doxorubicin, etoposide, 5-fluorouracil, hexamethamelemine, ifosamide, IL-2, interferon, lorazepam, misonidazole, mitotane, pamidronate, pentostatin, plicamycin, procarbazine, suramin, topotecan, and xeloda. These chemotherapeutic agents are used to treat a variety of cancers such as breast cancer, colon cancer, Hodgkin's disease, Kaposi's sarcoma, Letterer-Siwe disease, leukemia, lung cancer, lymphoma, melanoma, non-small-cell lung cancer, ovarian cancer, pancreatic cancer, stomach cancer, tumors, and uterine cancer.

Standard dosages for the therapeutic agents described herein are known in the art, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). One skilled in the art can determine the appropriate dosage for each therapeutic for a patient depending on such variables as the type and extent of the disorder, the overall health status of the particular patient, the formulation of the compound, and its route of administration. Standard clinical trials maybe used to optimize the dose and dosing frequency for any particular therapeutic agent.

In order for these drugs to provide the maximum therapeutic effect, a patient must be able to tolerate any side effects caused by the treatment. The neurotoxic side effects of these agents may, however, limit the amount of an agent that can be administered to particular patient. This limit may decrease the effectiveness of a treatment, thereby prolonging treatment or causing the ultimate failure of the treatment. In addition, even when a particular treatment is effective against a disease or condition, e.g., cancer, the neurotoxic side effects may remain after treatment has ceased. While some of these side effects spontaneously remit over time, some are permanent or last for months or years after treatment. Therefore, treatments designed to reduce neurotoxic side effects have the potential benefits of increasing survival by enabling longer treatments with particular agents and improving the quality of life during and after the treatment by alleviating the side effects.

To this end, we have discovered that extracts of Ginkgo biloba are useful in treating the neurotoxic side effects of therapeutic agents.

Ginkgo Extracts

Characterization. “Ginkgo extract” was originally described as a complex mixture containing flavonoid glycosides and several other substances which had been known to occur in the leaves of the Ginkgo tree. The basic difference between a herbal and a chemically-defined drag is that the former almost always contain a mixture of putative active substances and can be of widely varying quality and potency. The quality of the Ginkgo biloba leaves is important for producing a standardized Ginkgo extract.

The composition of the most well studied extract of Ginkgo biloba, EGb 761, is defined by the German Federal Health Authority as follows: “a dry extract from the dried leaves of Ginkgo biloba Linne manufactured using acetone/water and subsequent purification steps without additionally mixing concentrates or isolated active ingredients (DeFeudis FV 1998 Ginkgo biloba extract (EGb 761): from chemistry to clinic. Ullstein Medical, Wisbaden, Germany). EGb 761 has been commercialized in Europe under the names Tanakan™, Ginkgor™, Rokan™, Tebonin™ where it is available only under medical supervision (a prescription is required). In the U.S. there are many over-the-counter Ginkgo biloba supplements where EGb 761 is the basic ingredient, such as Ginkgoba™, GinkgoGold™, and Quatera™. EGb 761 quality and activity may be controlled by a well-defined, patented manufacturing process (European Patent No. 0431535 B1 and U.S. Pat. No. 5,399,348, hereby incorporated by reference). The herb:extract ratio is on average 50:1.

The EGb 761 extract is characterized as containing 24% ginkgo flavone glycosides and 6% terpene tri lactones (3.1% ginkgolides and 2.9% bilobalide). In addition, EGb 761 contains other substances (such as proanthocyanidins, carboxylic acids, non-flavone glucosides, and some high molecular weight components) whose concentration also depends upon the method of production and whose consistency is also important in ensuring high quality. EGb 761 is free of ginkgolic acids. Numerous studies have shown that several constituents of EGb 761 are individually biologically active and play a role in its pharmacological and therapeutic activities. It is believed, however, that it is the complementary and/or synergistic effects of these components that confer to the Ginkgo extract its regulatory and protective actions, making it an equilibrated “totum” (Drieu K, Jaggy H 1999 History, development and constituents of EGb 761. In: Ginkgo biloba. Van Beek T (ed). Medicinal and Aromatic Plants and Industrial Profiles Series, Harwood Academic Publishers, 267-277). Other Ginkgo extracts, e.g., IPS200 (a preparation of EGb 761 that lacks proanthocyanidins), LI1379, LI1370, BN 52063 (containing 40% ginkgolide A, 40% ginkgolide B, and 20% ginkgolide C), PN246 (containing 7 mg terpene lactones and 24 mg flavone glycosides per coated tablet; Bio-Biloba, Pharma Nord, Vejle, Denmark), Geriaforce®(a water/alcohol extract (70% v/v) of Ginkgo leaves, 1:4 extract; 0.34 mg/mL total ginkgolides and 0.20 mg/mL total flavone glycosides; Biohorma B.V.), and ZGE, are known in the art and may be useful in the methods described herein. Additional extracts may, for example, contain at least 5%, 10%, 15%, 20,%, 24%, 30%, 35%, 40%, 45%, 50%, 75%, 85%, or 95% of ginkgo flavone glycosides.

Therapeutic Activity. Numerous studies describe the beneficial effects of Ginkgo extracts on patients with disturbances in vigilance, memory, and cognitive functions associated with aging and senility, and on those with all types of dementias, mood changes, and deficiency in the ability to cope with daily stressors (DeFeudis FV 1998 Ginkgo biloba extract (EGb 761): from chemistry to clinic. Ullstein Medical, Wisbaden, Germany; Le Bars P L, Katz M M, Berman N, Itil T M, Freedman A M Schatzberg A F 1997 A placebo-controlled, double-blind, randomized trial of an extract of Ginkgo biloba for dementia. JAMA 278:1327-1332, Lemay M, Kergoat M-J, Lupien S 1999 Ginkgo biloba: what good is it? Mat. Medicine Canada 2:3437). In addition, there have been attempts to use Ginkgo extracts to improve the efficacy of cancer therapies employing chemotherapeutic agents such as 5-fluorouracil, doxetacel, doxorubicin, and adriamycin. The standardized extract of Ginkgo biloba leaves, EGb 761, has been used in most of these studies. In addition, EGb 761 has been shown to have cardioprotective effects. The most important active agents in EGb 761 are three major flavonoid groups whose chemical structures resemble those of nucleosides, isoalloxazine, and folic acid; and two terpene groups: a sesquiterpene, bilobalide; and diterpenes, the ginkgolides.

Activity Against Neurotoxicity. We have discovered that the administration of a Ginkgo extract is useful for treating the neurotoxic side effects of therapeutic agents. An exemplary Ginkgo extract for the treatments described herein is EGb 761. Other Ginkgo extracts are known in the art. Neurotoxic side effects that are treated according to methods described herein include, without limitation, pain, lack of mobility, ataxia, numbness, tingling, weakness in limbs, nystagmus, dizziness, dysmetria, dysarthria, dysdiadochokinesia, somnolence, seizure, altered personality, areflexia, constipation, hoarseness, orthostatic hypotension, gait disorders, stupor, coma, lethargy, confusion, depression, hallucinations, myoclonus, decreased vibratory sensation, decreased deep tendon reflex, hypersensitivity to temperature (e.g., hot or cold), and paresthesias.

A Ginkgo extract may be administered prior to and/or concurrently with a therapeutic agent in order to reduce or prevent any neurotoxicity associated with treatment with the agent. Alternatively or additionally, a Ginkgo extract may be administered after cessation of treatment with a therapeutic agent in order to treat the effects of neurotoxicity caused by the agent. Treatment with Ginkgo extract and therapeutic agents may also be alternated.

A Ginkgo extract may also be administered with other compounds that treat neurotoxicity such as antioxidants (e.g., amifostine, alpha-lipoic acid, sodium thiosulfate, diethyldithiocarbamate, 4-methylthiobenzoic acid, L- and D-methionine, salicylate, or glutathione), neurotrophic factors (e.g., nerve growth factor, neurotrophin-3, neurotrophin-4/5, brain-derived neurotrophic factor, and glial-derived neurotrophic factor), melanocortins (e.g., adrenocorticotropin (ACTH), alpha, beta and gamma-melanocyte-stimulating hormones, or Org2766), glutamate, calcium-magnesium infusions, antiepileptic drugs (e.g., carbamazepine or gabapentin), insulin-like growth factor I, or a combination thereof. Standard dosages for these other compounds may be found in the art, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In addition, one skilled in the art can determine the appropriate dosage for each therapeutic for a patient depending on such variables as the type and extent of the disorder, the overall health status of the particular patient, the formulation of the compound, and its route of administration. Standard clinical trials maybe used to optimize the dose and dosing frequency for any particular compound used to treat neurotoxicity.

Ginkgo extracts are desirably administered orally, but the method of administration may depend on the particular disease being treated or therapeutic agent being used. Alternative routes of administration include topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or by any other suitable route of administration.

The Ginkgo extract may also be administered with the therapeutic agent (e.g., oxaliplatin), and optionally other compounds for the treatment of neurotoxicity as described herein, in the same dosage unit, e.g., a bolus for injection or a pill. The entire formulation desirably will contain an amount of the therapeutic agent effective to treat the underlying disorder and an amount of the Ginkgo extract, and optional other compounds, effective to treat neurotoxicity caused by the therapeutic agent. Methods well known in the art for making formulations are found, for example, in Remington: The Science and Practice of Pharmacy (20th ed., A. R. Gennaro ed., Lippincott: Philadelphia, 2000). Pharmaceutically acceptable carriers for such formulations are also known in the art.

The Ginkgo extract can be administered to human patients in therapeutically effective amounts to provide therapy for neurotoxicity. Typical dose ranges are from 0.1 μg/kg to 1, 5, or 10 mg/kg of body weight per day, e.g., 0.5 mg/kg. An exemplary dosage is 120 mg of EGb 761 twice a day. The dosage of Ginkgo extract to be administered is likely to depend on such variables as the type and extent of the disorder, the overall health status of the particular patient, the formulation of the compound, and its route of administration. Standard clinical trials maybe used to optimize the dose and dosing frequency for any particular compound.

The level of neurotoxicity of a patient can be measured by standard physical examinations or by comparison of the patient history before and after treatment with a Ginkgo extract. For example, patients may be asked to complete questionnaires on their physical and mental condition before, during, and after treatment. In addition, patients may be subjected to standard cognitive or sensory tests to determine the level of neurotoxicity.

The following Example is merely intended to illustrate the invention and not to limit the invention in any way.

### EXAMPLE 1

We have tested the impact of a commercially available over-the-counter preparation of Ginkgo biloba, containing the standardized EGb 761 extract (Ginkgoba™) on patients receiving oxaliplatin. All patients were already receiving the oxaliplatin and had developed neurotoxicity prior to starting Ginkgo. Based on our data and the reported literature, we recommended Ginkgoba™ 120 mg twice a day, to be taken continuously (daily). The acute neurotoxicity was then measured by patient history prior to receiving Ginkgo (previous cycle of chemotherapy) and compared to that after treatment with Ginkgo (subsequent cycle of chemotherapy). We treated eight patients who were experiencing significant neurotoxicity from their treatments but were continuing to benefit from the anti-cancer actions of the oxaliplatin. Patients were treated continuously with doses of 120 mg of Ginkgoba™ twice a day for several months, during the treatment period and for one to two months after stopping the oxaliplatin. Seven of the eight patients reported a significant reduction in the neurotoxicity and an improvement in quality of life. Neurotoxicity was measured using patient history and physical examination. Five of the patients were men, three were women. All patients had advanced colon cancer, and all had developed some degree of neurotoxicity. The average age of the patients was approximately 55.

### OTHER EMBODIMENTS

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are within the claims.

What is claimed is:

1. A method of treating therapeutic-induced neurotoxicity in a patient, said method comprising administering to said patient a therapeutically effective amount of an extract of Ginkgo biloba.

2. The method of claim 1, wherein said neurotoxicity is induced by a therapeutic agent selected from the group consisting of an immunosuppressant, an antibiotic, an antiarrhythmic agent, an antilipidemic agent, a chemotherapeutic agent, or a combination thereof.

3. The method of claim 2, wherein said therapeutic agent is cyclosporine, tacrolimus, chloramphenicol, chloroquine, isoniazid, metronidazole, nitrofurantoin, caprolactam, rifampin, ethionamide, cycloserine, erythromycin, colistin, vancomycin, ethambutol, lincomycin, clindamycin, penicillin, imipenem, cefepime, ceftazidime, cefazolin, cefmetazole, benzylpenicillin, trovafloxacin, ciprofloxacin, levofloxacin, ofloxacin, amiodarone, bezafibrate, clofibrate, or a combination thereof.

4. The method of claim 2, wherein said therapeutic agent is a sulfonamide, tetracycline, aminoglycoside, tetracycline, polymyxin, beta lactam, carbapenem, cephalosporin, monobactam, fluoroquinolone, or a combination thereof.

5. The method of claim 2, wherein said chemotherapeutic agent is an alkylating agent, an antimetabolite agent, an antimicrotubule agent, an antimiotic agent, an antitumor antibiotic, or a combination thereof.

6. The method of claim 2, wherein said chemotherapeutic agent is L-asparaginase, carboplatin, chlorambucil, cytarabine, etoposide, hexamethamelemine, ifosamide, IL-2, interferon, lorazepam, misonidazole, mitotane, oxaliplatin, pamidronate, pentostatin, plicamycin, procarbazine, suramin, topotecan, vinblastine, vincristine, vindesine, vinorelbine, xeloda, JM216, ZD0473, BBR3464, SPI-77, nedaplatin, or a combination thereof.

7. The method of claim 2, wherein said chemotherapeutic agent is oxaliplatin.

8. The method of claim 1, wherein said neurotoxicity is induced by almitrine bimesylate, thalidomide, colchicine, disulfiram, phenyloin, dapsone, pyridoxine, sodium aurothiomalate, or a combination thereof.

9. The method of claim 1, wherein said extract comprises EGb 761, IPS200, LI1379, LI1370, BN 52063, PN246, Geriaforce®, or ZGE.

10. The method of claim 1, wherein said patient is suffering from breast cancer, colon cancer, Hodgkin's disease, Kaposi's sarcoma, Letterer-Siwe disease, leukemia, lung cancer, lymphoma, melanoma, ovarian, non-small-cell lung cancer, pancreatic cancer, stomach cancer, or uterine cancer.

11. The method of claim 10, wherein said patient is suffering from colon cancer.

12. The method of claim 1, further comprising administering an antioxidant, neurotrophic factor, melanocortin, glutamate, calcium-magnesium infusion, antiepileptic drug, insulin-like growth factor I, or a combination thereof.

13. The method of claim 12, wherein said antioxidant is amifostine, alpha-lipoic acid, sodium thiosulfate, diethyldithiocarbamate, 4-methylthiobenzoic acid, L- and D-methionine, salicylate, or glutathione.

14. The method of claim 12, wherein said antiepileptic drug is carbamazepine or gabapentin.

15. The method of claim 12, wherein said melancortin is adrenocorticotropin (ACTH), alpha, beta, or gamma-melanocyte-stimulating hormone, or Org2766.

16. The method of claim 12, wherein said neurotrophic factor is nerve growth factor, neurotrophin-3, neurotrophin-4/5, brain-derived neurotrophic factor, or glial-derived neurotrophic factor.

17. The method of claim 1, wherein said administering of said extract occurs before, during, or after, or a combination thereof, relative to administration of a therapeutic agent to said patient.

18. The method of claim 17, wherein said extract is administered concurrently with said therapeutic agent.

19. The method of claim 17, wherein said extract is administered prior to treatment with said therapeutic agent.

20. The method of claim 17, wherein said extract is administered after treatment with said therapeutic agent.

21. The method of claim 1, wherein administration of said extract is alternated with administration of a therapeutic agent.

22. The method of claim 1, wherein administration of said extract reduces said therapeutic-induced neurotoxicity.

23. The method of claim 1, wherein said neurotoxicity comprises pain, lack of mobility, ataxia, numbness, tingling, weakness in limbs, nystagmus, dizziness, dysmetria, dysarthria, dysdiadochokinesia, somnolence, seizure, altered personality, areflexia, constipation, hoarseness, orthostatic hypotension, gait disorders, stupor, coma, lethargy, confusion, depression, hallucinations, myoclonus, decreased vibratory sensation, decreased deep tendon reflex, hypersensitivity to temperature, paresthesias, or a combination thereof.

24. The method of claim 1, wherein prior to administering said extract to said patient, said patient is diagnosed with therapeutic-induced neurotoxicity.

25.-60. (canceled)
